(19) United States
(12) Patent Application Publication
(10) Pub. No.: US 2013/0210097 A1
(43) Pub. Date: Aug. 15, 2013

(54) GLYCOLIC ACID FERMENTATIVE PRODUCTION WITH A MODIFIED MICROORGANISM

(71) Applicants: Wanda Dischert, Vic-Le-Comte (FR); Cedric Colomb, Clermont-Ferrand (FR); Gwenaelle Bestel-Corre, Saint Beauzire (FR)

(72) Inventors: Wanda Dischert, Vic-Le-Comte (FR); Cedric Colomb, Clermont-Ferrand (FR); Gwenaelle Bestel-Corre, Saint Beauzire (FR)

(73) Assignee: METABOLIC EXPLORER, SAINT-BEAUZIRE (FR)

(21) Appl. No.: 13/817,067

(22) PCT Filed: Aug. 27, 2010
(86) PCT No.: PCT/IB2010/002545

§ 371 Date: Apr. 29, 2013

(51) Int. Cl. C12P 7/42; C12N 15/70

(52) U.S. Cl. CPC C12P 7/42; C12N 15/70
USPC 435/146; 435/252.33

(57) ABSTRACT

The present invention is related to a method for the fermentative production of glycolic acid, its derivatives or precursors, comprising the culture of an Escherichia coli strain in an appropriate culture medium comprising a carbon source, and the recovery of glycolic acid in the medium, wherein said E. coli strain is modified to improve the conversion of orotate into orotidine 5′-P.

The invention is also related to the modified E. coli strain, showing an improved conversion of orotate into orotidine 5′-P, and optionally that was furthermore modified for an improved glycolic acid production.

### OBJECT OF THE INVENTION

The present invention relates to an improved method for the biological production of glycolic acid from an inexpensive carbon substrate such as glucose or other sugars. The invention relates to the modification of E. coli K-12 genomic DNA, such that said microorganism comprises an increased orotate phosphoribosyl transferase activity (OPRTase), with the goal to reduce the production of the by-product orotate and to optimize glycolic acid synthesis.

### BACKGROUND OF THE INVENTION

Glycolic Acid (HOCH₂COOH), or glycolate, is the simplest member of the alpha-hydroxy acid family of carboxylic acids. Glycolic acid has dual functionality with both alcohol and moderately strong acid functional groups on a very small molecule. Its properties make it ideal for a broad spectrum of consumer and industrial applications, including use in water well rehabilitation, the leather industry, the oil and gas industry, the laundry and textile industry, and as a component in personal care products.

Glycolic Acid can also be used to produce a variety of polymeric materials, including thermoplastic resins comprising polyglycolic acid. Resins comprising polyglycolic acid have excellent gas barrier properties, and such thermoplastic resins comprising polyglycolic acid may be used to make packaging materials having the same properties (e.g., beverage containers, etc.). The polyester polymers gradually hydrolyze in aqueous environments at controllable rates. This property makes them useful in biomedical applications such as dissolvable sutures and in applications where a controlled release of acid is needed to reduce pH. Currently more than 15,000 tons of glycolic acid are consumed annually in the United states.

Although Glycolic Acid occurs naturally as a trace component in sugarcane, beets, grapes and fruits, it is mainly synthetically produced. Other technologies to produce Glycolic Acid are described in the literature or in patent applications. For instance, Mitsui Chemicals, Inc. has described a method for producing the said hydroxycarboxylic acid from aliphatic polyhydric alcohol having a hydroxyl group at the end by using a microorganism (EP 2 025 759 A1 and EP 2 025 760 A1). This method is a bioconversion as the one described by Michihiko Kataoka in its paper on the production of glycolic acid using ethylene glycol-oxidizing microorganisms (Biosci. Biotechnol. Biochem., 2001). Glycolic acid is also produced by bioconversion from glycolonitrile using mutant nitrilases with improved nitrilase activity and that technique was disclosed by Dupont de Nemours and Co in WO2006/069110. Methods for producing Glycolic Acid by fermentation from renewable resources using other bacterial strains were disclosed in patent applications from Metabolic Explorer (WO 2007/141316 and U.S. 61/162,712 and EP 09155971.6 filed on 24 Mar. 2009).

In their goal to build a better strain for producing glycolic acid, the inventors of the present invention have been interested in some specific E. coli strains.

Escherichia coli was the first and is still one of the most commonly used production microorganism in industrial biotechnology. Individual clones within the E. coli K-12 strain are particularly attractive hosts for the manipulations of recombinant DNA and the production of bulk chemicals due to the many years of research on this strain. The E. coli K-12 strains used for both research and commercial purposes today are derivatives of clones which were created and isolated in the first studies of this strain, by using irradiation with X-rays, and later with UV radiation to induce random mutations (Bachmann, B. J. 1987. Derivations and genotypes of some mutant derivatives of E. coli K-12, p. 1191-1219. In J. L. Ingraham, K. B. Low, B. Magasanik, M. Schaechter, and H. E. Humbarger (ed), Escherichia coli and Salmonella typhimurium: cellular and molecular biology). Some of the mutants or derivatives have evolved through purposeful selection and, thus, have well characterized mutations. It is, however, also recognized that many of the present day derivatives contain undetected and/or, as yet, uncharacterized allelic differences. Thus, members of the E. coli K-12 strain differ from one another by point mutations, in one or many genes.

Many E. coli K-12 strains have a frame shift mutation in the rph gene (Jensen K. F. 1993, J. Bacteria 175:3401-3707). This point mutation results in a frame shift of translation over the last 15 codons and reduces the size of the rph gene product by 10 amino acids residues. The truncated protein lacks Ribonuclease PH activity, and the premature translation stop in the rph cistron explains the low levels of orotate phosphoribosyltransferase in E. coli K-12, since close coupling between transcription and translation is needed to support optimal levels of transcription past the intercistronic pyrE attenuator.

This point mutation has been demonstrated to affect expression of the downstream pyrE gene encoding an orotate phosphoribosyl transferase (ORPTase) which catalyzes the transformation of orotic acid to orotidine 5′-phosphate (Poulsen P et al. 1984, EMBO 3:1783-1790). Since the expression of the pyrE gene is reduced, decreased levels of ORPTase result in accumulation of the substrate orotic acid in the cell and growth medium during cell growth (Womack J. E. and O'Donavan G. A. 1978, J. Bacteriol, 136:825-827).

Moreover, the patent U.S. Pat. No. 5,932,43 describes that the restoration of a wild type rph gene in E. coli K-12 strains containing the frame shift mutation increases the amount of heterologous protein produced in such strains.

Orotate is undesirable because it represents a consumption of carbon that could otherwise be used to generate biomass or glycolic acid. Moreover, orotate is a by-product difficult to eliminate during the purification of glycolic acid, and thus increases the purification cost. In addition, traces of orotate might colour the final product.

The problem solved by the present invention is decreasing the orotate accumulation during the biological production of glycolic acid from an inexpensive carbon substrate such as glucose or other sugars. The reduction of cost can be significant since the characteristics of glycolate production are improved.

### SUMMARY OF THE INVENTION

The present invention relates to a process for improving the fermentative production of glycolic acid by an E. coli strain, wherein said strain has been modified to improve the conversion of orotate into orotidine 5′-Phosphate. Increasing said conversion has an effect on the production of glycolic acid, that is improved. The method for the fermentative production of glycolic acid, its derivatives or precursors, comprises the culture of an Escherichia coli strain in an appropriate culture medium comprising a carbon source, and the recovery of glycolic acid in the medium, wherein said strain is modified to improve the conversion of orotate into orotidine 5′-Phosphate.

In a first embodiment of the invention, the orotate phosphoribosyl transferase (OPRTase) specific activity is increased in the modified strain.

In another embodiment of the invention, the E. coli strain is modified to enhance the production of phosphoribosyl pyrophosphate (PRPP), an essential cofactor of the reaction converting orotate into orotidine 5′-phosphate.

Both modifications, increase of the OPRTase activity and increase of the production of PRPP, can be introduced into the same E. coli strain.

In a preferred embodiment of the invention, the strain is furthermore genetically engineered to enhance the production of glycolic acid.

The invention is also related to a method for preparing glycolic acid wherein the microorganism according to the invention is grown in an appropriate growth medium comprising a source of carbon, and glycolic acid is recovered.

The invention is also related to a modified E. coli strain, presenting the modifications such as described above.

### BRIEF DESCRIPTION OF THE DRAWINGS

FIG. 1: Pyrimidine biosynthesis and pentose phosphate pathway involving the enzymes PyrE (orotate phosphoribosyl-transferase) and PrsA (PRPP synthetase).

FIG. 2: Schematic illustration showing the connexions between the three different biosynthesis pathways: glycolate, pentose phosphate and pyrimidine pathways.

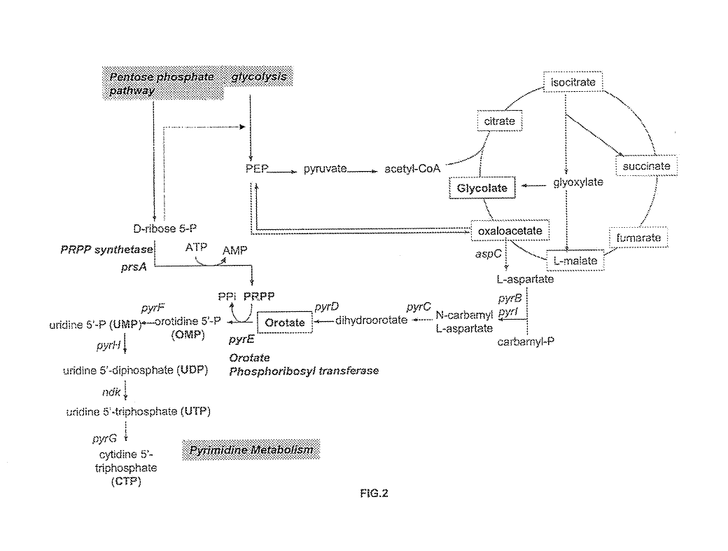

FIG. 3: Map of the plasmid pBBR1MCS5-Ptrc04/RBS01*5-pyrE-TTs.

FIG. 4: Map of the plasmid pBBR1MCS5-Ptrc04/RBS01*5-pyrE-prsA-TTs.

### DETAILED DESCRIPTION

The present invention relates to a novel method for the fermentative production of glycolic acid, its derivatives or precursors, comprising the culture of an Escherichia coli strain in an appropriate culture medium comprising a source of carbon, and the recovery of glycolic acid in the medium, said E. coli strain being modified to improve the conversion of orotate into orotidine 5′-Phosphate.

In a preferred embodiment of the invention, the production of glycolic acid is also improved in the E. coli strain modified to improve the conversion of orotate into orotidine 5′-Phosphate.

In the present invention, the terms “glycolate” and “glycolic acid” are used interchangeably.

The term “glycolic acid, its derivatives or precursors” designates all intermediate compounds in the metabolic pathway of formation and degradation of glycolic acid. Precursors of glycolic acid are in particular: citrate, isocitrate, glyoxylate, and in general all compounds of the glyoxylate cycle. Derivatives of glycolic acid are in particular glycolate esters such as ethyl glycolate ester, methyl glycolate ester and polymers containing glycolate such as polyglycolic acid.

According to the invention, the terms “fermentative production', ‘fermentation’ or ‘culture” are used interchangeably to denote the growth of bacteria on an appropriate growth culture medium, comprising a carbon source, wherein the carbon source is used both and concomitantly for the growth of the strain and for the production of the desired product, glycolic acid.

An “appropriate culture medium” is a medium appropriate for the culture and growth of the microorganism. Such media are well known in the art of fermentation of microorganisms, depending upon the microorganism to be cultured. The appropriate culture medium comprises “a source of carbon” which refers to any carbon source capable of being metabolized by a microorganism.

In relation with the present invention, “being metabolized” is understood in its general meaning of transformation of energy and matter allowing growth of the microorganism, or at least maintain life.

In the fermentative process of the invention, the source of carbon is used for:
biomass production—growth of the microorganism by converting inter alia the carbon source of the medium, and,
glycolic acid production—transformation of the same carbon source into glycolic acid by the same biomass.

The two steps are concomitant and the transformation of the source of carbon by the microorganism to grow results in the glycolic acid secretion in the medium, since the microorganism comprises a metabolic pathway allowing such conversion.

The source of carbon is selected among the group consisting of glucose, sucrose, monosaccharides (such as fructose, mannose, xylose, arabinose), oligosaccharides (such as galactose, cellobiose . . . ), polysaccharides (such as cellulose), starch or its derivatives, glycerol and single-carbon substrates. An especially preferred carbon source is glucose. Another preferred carbon source is sucrose.

The terms “improved”, “increased”, “increase” or “improve” mean that the amount of conversion of orotate into orotidine 5′-Phosphate is higher in the modified microorganism compared to the corresponding unmodified microorganism. Said conversion can be improved by different means, and in particular by:
the increase of the quantity of the initial substrate (orotate),
the increase of the availability of the cofactor (PRPP),
the increase of the activity of the enzyme catalyzing the reaction (Orotate phosphoribosyl transferase).

In a specific aspect of the invention, the strain has an increased orotate phosphoribosyl transferase specific activity. Orotate phosphoribosyl transferase or “OPRTase” is an enzyme catalyzing the conversion of orotate into orotidine 5′-Phosphate (OMP).

In particular, the strain exhibits an increased orotate phosphoribosyl transferase specific activity of about 30 units, preferably at least 50 units and most preferably at least 70 units.

In a preferred aspect of the invention, the expression of the gene pyrE encoding the orotate phosphoribosyl transferase enzyme is increased.

The term “expression” refers to the transcription and translation from a gene to the protein, product of the gene.

The gene expression can be increased by various means such as:
expression of an heterologous gene on a plasmid, introduced into the strain;
overexpression of the endogenous gene, obtained by replacement of the endogenous promoter with a stronger promoter, or by increasing the number of copy of the genes on the chromosome;
expression of the gene from an artificial promoter at another locus or other loci on the chromosome.

In a more preferred aspect of the invention, the expression of the gene pyrE is restored, in an E. coli K12 strain having a frameshift mutation in the rph-pyrE operon.

The nucleotide sequence of an rph gene containing a frame shift mutation is set forth by Jensen, K. F. (1993). Additionally, the nucleotide sequence of the wild type rph-pyrE operon is available from the GenBank/EMBL data bank under accession numbers X00781 and X01713, and the sequence of the intercistronic rph-pyrE segment and the flanking regions is available from the EMBL data bank under accession number X72920. It is also understood by those skilled in the art that, referring to wild-type rph and pyrE DNA sequences, such sequences include natural and synthetic sequences which are functionally equivalent to those published or deposited.

The term “E. coli K-12 strain” is understood to include the culture Escherichia coli from the collection of the bacteriology department at Stanford University and all derivatives of Lederberg strain W1485, which arose from the original E. coli K-12 strain after treatment with UV light, X-rays and/or other chemical or genetic treatments (Bachmann, B. J. 1987. Derivations and genotypes of some mutant derivatives of Escherichia coli K-12, p. 1191-1219. In J. L. Ingraham, K. B. Low, B. Magasanik, M. Schaechter, and H. E. Umbarger (ed.), Escherichia coli and Salmonella typhinurium: cellular and molecular biology. American Society for Microbiology, Washington, D.C).

The terms “E. coli K12 strain having a frameshift mutation in the rph-pyrE operon” refers to E. coli strain derivatives of the Lederberg strain W1485, bearing a known point mutation on the rph gene. E. coli strains missing a ‘CG’ bases pair from a block of 5 ‘GC’ found 43 to 47 pairs of bases upstream of the rph stop codon, are considered as mutant strains compared to those bearing a non mutated, wild-type rph gene (Jensen K, 1993, J. Bacteriol. 175:3401-3407).

It has been previously demonstrated that the frame shift mutation in the rph gene of E. coli K-12 strains has a polar effect on the expression of the pyrE gene, located downstream of rph, in a common “operon”. Therefore a mutation in the rph gene results in a low level of orotate phosphoribosyl transferase and as a consequence, in accumulation of orotic acid.

E. coli K-12 strains with the mutated rph-pyrE operon produce orotate phosphoribosyltransferase enzyme (PyrE) with a specific activity of about 5 to 20 units, while other E. coli strains with a wild-type rph-pyrE operon, in other words with a wild-type pyrE expression, exhibit OPRTase specific activity levels of about 30 to 90 units.

Accumulation of orotic acid in strains having the frame shift mutation on rph might interfere with the production, the isolation and the purification of glycolate. Thus, by significantly diminishing this orotic acid accumulation in E. coli K-12 which exhibits wild-type OPRT activity (specific activity of at least 30 units), the production of glycolic acid could be significantly improved.

The term “restoration” refers to the specific genetic alterations or manipulations, known by the man skilled in the art, used to recreate the wild-type rph-pyrE operon.

In this specific case, one possibility to increase the transcription of pyrE is to restore the wild-type sequence of the rph-pyrE operon by correcting the point mutation in rph responsible for the poor transcription of pyrE.

E. coli K-12 strains that possess a wild-type operon, can be identified by determining the levels of the orotate phosphoribosyltransferase activity and/or by sequencing the rph-pyrE region contained therein.

When referring to “the yield”, “the level” or “the amount” of a chemical compound, these terms are understood to mean a quantitative amount of an essentially pure product. Conventional chemical detection methods such as GCMS, HPLC, spectro-photometric techniques, and enzymatic activity can be used.

In the present invention, enzymes are identified by their specific activities. This definition thus includes all polypeptides that have the defined specific activity also present in other organisms, more particularly in other microorganisms. Enzymes with similar activities can be identified by homology to certain families defined as PFAM or COG.

PFAM (protein families' database of alignments and hidden Markov models; http://www.sanger.ac.uk/Software/Pfam/) represents a large collection of protein sequence alignments. Each PFAM makes it possible to visualize multiple alignments, see protein domains, evaluate distribution among organisms, gain access to other databases, and visualize known protein structures.

COGs (clusters of orthologous groups of proteins; http://www.ncbi.nlm.nih.gpv/COG/) are obtained by comparing protein sequences from 43 fully sequenced genomes representing 30 major phylogenic lines. Each COG is defined from at least three lines, which permits the identification of former conserved domains.

The means of identifying homologous sequences and their percentage homologies are well known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw) or MULTALIN (http://prodes.toulouse.inra.fr./multalin/cgi-bin/multalin.pl), with the default parameters indicated on those websites.

Using the references given in GenBank for known genes, those skilled in the art are able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well known to those skilled in the art, and are described, for example, in Sambrook et al. (1989 Molecular Cloning: a Laboratory Manual. 2^{nd} ed. Cold Spring Harbor Lab., Cold Spring Harbor, N.Y.).

In a specific embodiment of the invention, the strain presents an increased availability of 5-Phosphoribosyl 1-pyrophosphate (PRPP).

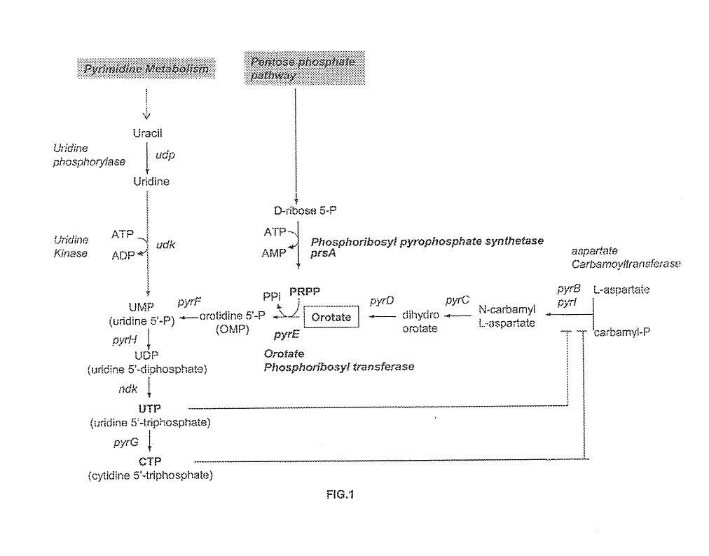

The terms “phosphoribosyl pyrophosphate”, “5-phosphoribose 1-pyrophosphate” and “PRPP” are used interchangeably. PRPP is a pentose phosphate formed from ribose 5-phosphate and one ATP (see on FIG. 1) by the enzyme phosphoribosyl pyrophosphate synthetase encoded by the gene prsA.

Phosphoribosyl pyrophosphate synthetase is involved in the first step of the biosynthesis of purine, pyrimidine, and nicotinamide nucleotides and in the biosynthesis of histidine and tryptophan (EP1529839A1 and EP1700910A2 from Ajinomoto).

The molecule PRPP is also an essential cofactor for the reaction catalyzed by the enzyme OPRTase (see above). Indeed, the reaction uses a pentose phosphate moiety from PRPP.

The term ‘increased availability’ means that PRPP is present in a higher quantity compared to an unmodified strain: either the production of PRPP is increased, either its consumption is decreased.

In a particular aspect of the invention, the expression of the gene prsA encoding the phosphoribosylpyrophosphate synthase is increased, therefore the production of PRPP is increased compared to an unmodified strain.

Various methods are useful to increase the expression of a gene and they are known by the man skilled in the art:
Expression of the gene from a plasmid DNA,
Replacement of the natural promoter of the gene by a strong promoter directly on the chromosome,
Expression of the gene from an artificial promoter at another locus or other loci on the chromosome.

In another embodiment of the invention, the strain is further modified to enhance the production of glycolic acid.

In particular, the modified microorganism might comprise at least one of the following modifications:
decrease of the conversion of glyoxylate to products other than glycolate, obtained in particular by the attenuation of the genes aceB, glcB, gcl, eda,
unability to substantially metabolize glycolate, obtained in particular by the attenuation of the genes glcDEFG, aldA,
increase of the glyoxylate pathway flux, obtained in particular by the attenuation of the genes icd, aceK, pta, ackA, poxB, iclR or fadR, and/or by the overexpression of the gene aceA,
increase of the conversion of glyoxylate to glycolate, obtained in particular by the overexpression of the genes ycdW or yiaE,
increase of the availability of NADPH, obtained in particular by the attenuation of the genes pgi, udhA, edd.

In particular, the microorganism is modified to have a low capacity of glyoxylate conversion, except to produce glycolate, due to the attenuation of the expression of genes encoding for enzymes consuming glyoxylate, a key precursor of glycolate:
aceB and gclB genes encoding malate synthases,
gcl encoding glyoxylate carboligase and
eda encoding 2-keto-3-deoxygluconate 6-phosphate aldolase.

Various methods are useful for the attenuation of the expression of genes:
Introduction of a mutation into the gene, decreasing the expression level of this gene,
Replacement of the natural promoter of the gene by a weak promoter, resulting in a lower expression,
Deletion of the gene if no expression is needed.

In a further embodiment of the invention, the E. coli K12 strain is modified in such a way that it is unable to substantially metabolize glycolate. This result can be achieved by the attenuation of at least one of the genes encoding for enzymes consuming glycolate:
glcDEF encoding glycolate oxidase, and
aldA encoding glycoaldehyde dehydrogenase.

Attenuation of genes can be done by replacing the natural promoter by a low strength promoter or by elements destabilizing the corresponding messenger RNA or the protein. If needed, complete attenuation of the gene can also be achieved by a deletion of the corresponding DNA sequence.

In another embodiment, the E. coli K12 strain according to the invention is transformed to increase the glyoxylate pathway flux.

The flux in the glyoxylate pathway may be increased by different means, and in particular:
i) decreasing the activity of the enzyme isocitrate dehydrogenase, encoded by the icd gene,
ii) decreasing the activity of at least one of the following enzymes:
   phospho-transacetylase, encoded by the pta gene
   acetate kinase, encoded by the ack gene
   pyruvate oxidase, encoded by the poxB gene
   Icd kinase-phosphatase, encoded by the aceK gene
iii) increasing the activity of the enzyme isocitrate lyase, encoded by the aceA gene.
   Decreasing the level of isocitrate dehydrogenase can be accomplished by introducing artificial promoters that drive the expression of the icd gene, coding for the isocitrate dehydrogenase, or by introducing mutations into the icd gene that reduce the enzymatic activity of the protein.
   Since the activity of the protein Icd is reduced by phosphorylation, it may also be controlled by introducing mutant aceK genes that have increased kinase activity or reduced phosphatase activity compared to the wild type AceK enzyme.
   Increasing the activity of the isocitrate lyase can be accomplished either by attenuating the level of iclR or fadR genes, coding for glyoxylate pathway repressors, or by stimulating the expression of the aceA gene, for example by introducing artificial promoters that drive the expression of the gene, or by introducing mutations into the aceA gene that increase the activity the encoded protein.

In another embodiment of the invention, the E. coli K12 strain contains at least one gene encoding a polypeptide catalyzing the conversion of glyoxylate to glycolate. In a preferred manner, the expression of the gene is increased.

In particular, this polypeptide is a NADPH dependent glyoxylate reductase enzyme that converts, the toxic glyoxylate intermediate into glycolate.

Preferably, said gene is chosen among the ycdW or yiaE genes from the genome of E. coli MG1655. If needed a high level of NADPH-dependant glyoxylate reductase activity can be obtained from chromosomally encoded genes by using one or several copies on the genome that can be introduced by methods of recombination known to the expert in the field. For extra chromosomal genes, different types of plasmids that differ with respect to their origin of replication and thus their copy number in the cell can be used. They may be present as 1-5 copies, ca 20 or up to 500 copies corresponding to low copy number plasmids with tight replication (pSC101, RK2), low copy number plasmids (pACYC, pRSF1010) or high copy number plasmids (pSK bluescript II). The ycdW or yiaE genes may be expressed using promoters with different strength that need or need not to be induced by inducer molecules. Examples are the promoters Ptrc, Ptac, Plac, the lambda promoter cI or other promoters known to the expert in the field. Expression of the genes may also be boosted by elements stabilizing the corresponding messenger RNA (Carrier and Keasling (1998) Biotechnol. Prog. 15, 58-64) or the protein (e.g. GST tags, Amersham Biosciences).

The gene encoding said polypeptide can be either exogenous or endogenous, and can be expressed chromosomally or extra-chromosomally.

In another embodiment of the invention, the E. coli K12 strain presents an increased NADPH availability for the NADPH-dependant glyoxylate reductase, which provides a better yield of glycolate production. This modification of the microorganism can be obtained through the attenuation of at least one of the genes selected among the following:
pgi encoding the glucose-6-phosphate isomerase,
udhA encoding the soluble transhydrogenase and
edd encoding the 6-phosphogluconate dehydratase activity.
   With such genetic modifications, all the glucose-6-phosphate will have to enter glycolysis through the pentose phosphate pathway and 2 NADPH will be produced per glucose-6-phosphate metabolized.

In a preferred embodiment of the invention, the modified microorganism comprise attenuation of the genes aceB, glcB, gcl, eda, glcDEFG, aldA, icd, aceK, pta, ackA, poxB, iclR and overexpression of the genes aceA and ycdW. Optionally the modified microorganism could also comprise attenuation of the genes pgi, udhA, and edd.

In an embodiment of the invention, the carbon source is chosen among the following group: glucose, sucrose, mono- or oligosaccharides, starch or its derivatives or glycerol, and combinations thereof.

The invention previously described is also related to a method for the fermentative preparation of glycolic acid comprising the following steps:
a) Fermentation of the microorganism producing glycolic acid,
b) Concentration of glycolic acid in the bacteria or in the medium and,
c) Isolation of glycolic acid from the fermentation broth and/or the biomass optionally remaining in portions or in the total amount (0-100%) in the end product.
   In a particular embodiment, the glycolic acid is isolated through a step of polymerization to at least glycolate dimers and recovered by depolymerization from glycolate dimers, oligomers and/or polymers.

Those skilled in the art are able to define the culture conditions for the microorganisms according to the invention. In particular the E. coli K12 strains are fermented at a temperature between 30° C. and 37° C.

The fermentation is generally conducted in fermenters with an inorganic culture medium of known defined composition adapted to the bacteria used, containing at least one simple carbon source, and if necessary a co-substrate necessary for the production of the metabolite.

The invention is also related to an E. coli K-12 strain with enhanced conversion of orotate into orotidine 5′-Phosphate.

In particular, said strain presents an increased orotate phosphoribosyl transferase specific activity.

In a preferred aspect of the invention, the expression of the gene pyrE encoding the orotate phosphoribosyl transferase enzyme is increased in said strain.

In a specific aspect of the invention, the strain is modified in the way that the expression of the gene pyrE is restored in an E. coli K12 strain having a frameshift mutation in the rph-pyrE operon.

In another aspect of the invention, the strain presents an increased availability of 5-Phosphoribosyl 1-pyrophosphate (PRPP).

In particular, the invention concerns an E. coli strain, wherein both the expression of gene pyrE and the production of PRPP are increased.

More specifically, the invention concerns a E. coli strain, wherein the gene prsA encoding the phosphoribosylpyrophosphate synthase as described above is overexpressed.

In another embodiment of the invention, the modified E. coli strain is furthermore modified to produce glycolic acid with high yield. In particular, said E. coli strain comprises at least one of the following modifications:
decrease of the conversion of glyoxylate to products other than glycolate, obtained in particular by the attenuation of the genes aceB, glcB, gcl, eda,
unability to substantially metabolize glycolate, obtained in particular by the attenuation of the genes glcDEFG, aldA,
increase of the glyoxylate pathway flux, obtained in particular by the attenuation of the genes icd, aceK, pta, ackA, poxB, iclR or fadR, and/or by the overexpression of the gene aceA,
increase of the conversion of glyoxylate to glycolate, obtained in particular by the overexpression of the genes ycdW or yiaE,
increase of the availability of NADPH, obtained in particular by the attenuation of the genes pgi, udhA, edd.

This microorganism is preferentially an E. coli K-12 strain, possessing an rph frame shift mutation [see Machida, H. and Kuninaka, A. (1969) and “Escherichia coli and Salmonella typhimurium: Cellular and Molecular Biology 1987], first corrected to contain at least a wild-type OPRT activity and then genetically engineered, in particular to avoid any conversion of glyoxylate to products other than glycolate.

Such strains can be identified by different methods already described in here; by measuring the OPRT activity, by DNA sequence analysis of the rph-pyrE operon and/or by checking the level of orotate accumulation.

### EXAMPLES

Several protocols were used to build the strains producing glycolic acid described in the following examples. The protocols are detailed below.

Protocol 1: Introduction of a PCR Product for Recombination and Selection of the Recombinants (Cre-LOX System)

The oligonucleotides chosen and given in Table 1 for replacement of a gene or an intergenic region were used to amplify either the chloramphenicol resistance cassette from the plasmid loxP-cm-loxP (Gene Bridges) or the neomycin resistance cassette from the plasmid loxP-PGK-gb2-neo-loxP (Gene Bridges). The PCR product obtained was then introduced by electroporation into the recipient strain bearing the plasmid pKD46 in which the system λ Red (γ, β, exo) expressed greatly favours homologous recombination. The antibiotic-resistant transformants were then selected and the insertion of the resistance cassette was checked by PCR analysis with the appropriate oligonucleotides given in Table 2.

Protocol 2: Transduction of Gene Deletions Using Phage P1

DNA transfer from one E. coli strain to another was performed by the technique of transduction with phage P1. The protocol was carried out in two steps, (i) the preparation of the phage lysate on the donor strain with a single modified gene and (ii) the transduction of the recipient strain by this phage lysate.

Preparation of the Phage Lysate
Seeding with 100 μl of an overnight culture of the strain MG1655 with a single modified gene of 10 ml of LB+Cm 30 μg/ml/Km 50 μg/ml+glucose 0.2%+CaCl₂ 5 mM.
Incubation for 30 min at 37° C. with shaking.
Addition of 100 μl of phage lysate P1 prepared on the donor strain MG1655 (approx. 1×10⁹ phage/ml).
Shaking at 37° C. for 3 hours until all cells were lysed.
Addition of 200 μl of chloroform, and vortexing.
Centrifugation for 10 min at 4500 g to eliminate cell debris.
Transfer of the supernatant into a sterile tube and addition of 200 μl of chloroform.
Storage of the lysate at 4° C.

Transduction
Centrifugation for 10 min at 1500 g of 5 ml of an overnight culture of the E. coli recipient strain in LB medium.

Suspension of the cell pellet in 2.5 ml of MgSO₄ 10 mM, CaCl₂ 5 mM.
Control tubes: 100 μl cells
   100 μl phages P1 of the strain MG1655 with a single gene deleted.
Tube test: 100 μl of cells+100 μl phages P1 of strain MG1655 with a single modified gene.
Incubation for 30 min at 30° C. without shaking.
Addition of 100 μl sodium citrate 1 M in each tube, and vortexing.
Addition of 1 ml of LB.
Incubation for 1 hour at 37° C. with shaking.
Plating on dishes LB+Cm 30 μg/ml/Km 50 μg/ml after centrifugation of tubes for 3 min at 7000 rpm.
Incubation at 37° C. overnight.

The antibiotic-resistant transformants were then selected and the insertion of the deletion was checked by PCR analysis with the appropriate oligonucleotides given in Table 2.

TABLE 1
Oligonucleotides used for the constructions described in the following examples

| | | | Homology | |
|---|---|---|---|---|
| | | | with | |
| | | SEQ | chromosoma | |
| | Name of | ID | 1 region | |
| Gene | oligo | N° | (Ecogene) | Sequence |
| rph + pyre | Oag | N° 1 | 3813155- | CGCCAAACTCTTCGCGATAGGCCTTAACCGCCGCCAGATG |
| | 0119_Dp | | 3813234 | TTCCGCCATTTCCGGCTTCTCTTCCAGGTAAGCAATCAGG |
| | yrE- | | | TAATACGACTCACTATAGGG |
| | loxP R | | | |
| | Oag | N° 2 | 3814543- | GGTGCGTCCCGTTACCCTGACTCGTAACTATACAAAACAT |
| | 0143_Dr | | 3814462 | GCAGAAGGCTCGGTGCTGGTCGAATTTGGCGATACCAAAG |
| | ph- | | | TGAATTAACCCTCACTAAAGGG |
| | loxP F | | | |
| pBBR1MC | Ptrc04/ | N° 3 | 3813791- | GATATCTTGACCATTAATCATCCGGCTCGTATAATGTGTG |
| S5- | RBS01*5- | | 3813764 | GAATAAGGAGGTATACTATGAAACCATATCAGCGCCAGTT |
| Ptrc04/ | pyrE F | | | TATTG |
| RBS01*5- | | | | Ptrc promoter and beginning of pyrE |
| pyrE- | pyrE R | N° 4 | 3813150- | GGTACCTTAAACGCCAAACTCTTCGCG |
| TTs | | | 3813170 | End of pyre |
| pBBR1MC | Oag | N° 5 | 1261119- | CCAGGTACCGCATGCCTGAGGTTCTTCTC |
| S5- | 0371- | | 1261099 | Beginning of prsA (ribosome binding |
| Ptrc04/ | prsA F | | | site) |
| RBS01*5- | KpnI | | | |
| pyrE- | Oag | N° 6 | 1260129- | CGGGTCTTTGACCCGGGTTCGA |
| prsA- | 0372- | | 1260150 | Sequence was modified to introduce a |
| TTs | prsA R | | | Smal restriction site |
| | SmaI | | | |

TABLE 2
Oligonucleotides used for checking the insertion of a
resistance cassette or the loss of a resistance cassette

| | | | Homology with | |
|---|---|---|---|---|
| | Names of | SEQ ID | chromosomal | |
| Gene | oligos | N° | region | Sequences |
| rph + pyrE | Oag | N° 7 | 3814843- | CGACAGGTTCAAGGCTACGG |
| | 0144_rph- | | 3814824 | |
| | loxP F | | | |
| | Oag | N° 8 | 3812969- | CACCACCGATGAAACCCTGC |
| | 0122_DpyrE | | 3812988 | |
| | R | | | |

### Example 1

### Genetic Reconstruction of the rph-pyrE Operon in the E. coli K-12 Strain Producing Glycolic Acid by Fermentation: MG1655 Ptrc50/RBSB/TTG-icd::Cm rph+pyrErc ΔaceB Δgcl ΔglcDEFGB ΔaldA ΔiclR Δedd+eda (pME101-ycdW-TT07-PaceA-aceA-TT01)

The strain E. coli MG1655 Ptrc50/RBSB/TTG-icd::Cm ΔaceB Δgcl ΔglcDEFGB ΔaldA ΔiclR Δedd+eda (pME101-ycdW-TT07-PaceA-aceA-TT01) was constructed according to the description given in patent application EP 2 027 277, and non published application EP 09155971.

E. coli wild type MG1655 strain has a frameshift mutation in the rph gene. To restore the orotate phosphoribosyltransferase activity level in the cell, the functional rph gene has been introduced in several steps into the strain E. coli MG1655 Ptrc50/RBSB/TTG-icd::Cm ΔaceB Δgcl ΔglcDEFGB ΔaldA ΔiclR Δedd+eda (pME101-ycdW-TT07-PaceA-aceA-TT01) to give E. coli MG1655 Ptrc50/RBSB/TTG-icd::Cm Δrph+pyrE::Nm ΔaceB Δgcl ΔglcDEFGB ΔaldA ΔiclR Δedd+eda (pME101-ycdW-TT07-PaceA-aceA-TT01).

Abbreviations:

Rph-pyrErc designates “reconstruction of rph-pyrE operon with a wild-type copy of rph”. The expression of pyrE is increased in such case.
Δrph+pyrE::Nm designates “deletion of the operon”.
When nothing is mentioned in the genotype, the operon is the same than in MG1655 E. coli K-12 strain, i.e. with a mutation in the rph gene.
1. Construction of the Strain MG1655 drph+pyrE::Nm

To delete the rph+pyrE region in the strain E. coli MG1655, the homologous recombination strategy described by Datsenko & Wanner (2000) was used. The construction is performed according to the technique described in the Protocol 1 with the respective oligonucleotides Oag 0119-DpyrE-loxP R and Oag 0143_Drph-loxP F (Seq. N°1 and N°2) given in table 1.

Oag 0119_DpyrE-loxP R
(SEQ ID NO 1)
CGCCAAACTCTTCGCGATAGGCCTTAACCGCCGCCAGATGTTCCGCCAT
TTCCGGCTTCTCTTCCAGGTAAGCAATCAGGTAATACGACTCACTATAG
GG

with
a region (upper case) homologous to the sequence (3813155-3813234) of the region pyrE (reference sequence on the website http://ecogene.org/),
a region (upper bold case) for the amplification of the neomycin resistance cassette (reference sequence Gene Bridges),

Oag 0143_Drph-loxP F
(SEQ ID NO 2)
GGTGCGTCCCGTTACCCTGACTCGTAACTATACAAAACATGCAGAAGGC
TCGGTGCTGGTCGAATTTGGCGATACCAAAGTGAATTAACCCTCACTAA
AGGG

with
a region (upper case) homologous to the sequence (3814543-3814462) of the region rph (reference sequence on the website http://ecogene.org/),
a region (upper bold case) for the amplification of the neomycin resistance cassette (reference sequence Gene Bridges).

The resulting PCR product was introduced by electroporation into the strain MG1655 (pKD46). The neomycin resistant transformants were then selected, and the insertion of the resistance cassette was verified by PCR analysis with the oligonucleotides Oag 0144_rph-loxP F and Oag 0122_DpyrE R defined in Table 2 (Seq. N°7 and N°8). The resulting strain was named MG1655 Δrph+pyrE::Nm.

2. Construction of the Strain E. coli MG1655 Ptrc50/RBSB/TTG-icd::Cm rph+pyrErc ΔaceB Δgcl ΔglcDEFGB ΔaldA ΔiclR Δedd+eda (pME101-ycdW-TT07-PaceA-aceA-TT01).

Firstly strain E. coli MG1655 Ptrc50/RBSB/TTG-icd::Cm Δrph+pyrE::Nm ΔaceB Δgcl ΔglcDEFGB ΔaldA ΔiclR Δedd+eda was constructed by the technique of transduction with phage P1 described in protocol 1. The donor strain was strain MG1655 Δrph+pyrE::Nm described above. The receiver strain E. coli MG1655 Ptrc50/RBSB/TTG-icd::Cm ΔaceB Δgcl ΔglcDEFGB ΔaldA ΔiclR Δedd+eda was described in previous patent applications mentioned above. Neomycine and chloramphenicol resistant transformants were selected and the insertion of the Δrph+pyrE::Nm region was verified by a PCR analysis with the oligonucleotides Oag 0144_rph-loxP F and Oag 0122_DpyrE R. The resulting strain was named MG1655 Ptrc50/RBSB/TTG-icd::Cm Δrph+pyrE::Nm ΔaceB Δgcl ΔglcDEFGB ΔaldA ΔiclR Δedd+eda.

To restore the functional rph gene, the strain E. coli MG1655 Ptrc50/RBSB/TTG-icd::Cm rph+pyrErc ΔaceB Δgcl ΔglcDEFGB ΔaldA ΔiclR Δedd+eda was constructed by the technique of transduction with phage P1 described in protocol 1. The donor strain is the CGSC #5073 strain (which can be obtained from the “E. coli Genetic Stock Center”, stock #5073, Yale University, New Haven, Conn.), with a wild-type rph gene (written herein as rph+pyrErc). Chloramphenicol resistant transformants were then selected for pyrimidine prototrophy and the insertion of the rph+pyrE region was verified by a PCR analysis with the oligonucleotides Oag 0144_rph-loxP F and Oag 0122_DpyrE R defined above. The resulting strain was validated by sequencing. The strain retained is designated MG1655 Ptrc50/RBSB/TTG-icd::Cm rph+pyrErc ΔaceB Δgcl ΔglcDEFGB ΔaldA ΔiclR Δedd+eda.

The plasmid pME101-ycdW-TT07-PaceA-aceA-TT01 was then introduced by electroporation in the strain designated MG1655 Ptrc50/RBSB/TTG-icd::Cm rph+pyrErc ΔaceB Δgcl ΔglcDEFGB ΔaldA ΔiclR Δedd+eda. The resulting strain MG1655 Ptrc50/RBSB/TTG-icd::Cm rph+pyrErc ΔaceB Δgcl ΔglcDEFGB ΔaldA ΔiclR Δedd+eda (pME101-ycdW-TT07-PaceA-aceA-TT01) was named AG0843.

### Example 2

### Construction of the Plasmid pBBR1MCS5-Ptrc04/RBS01*5-pyrE-TTs

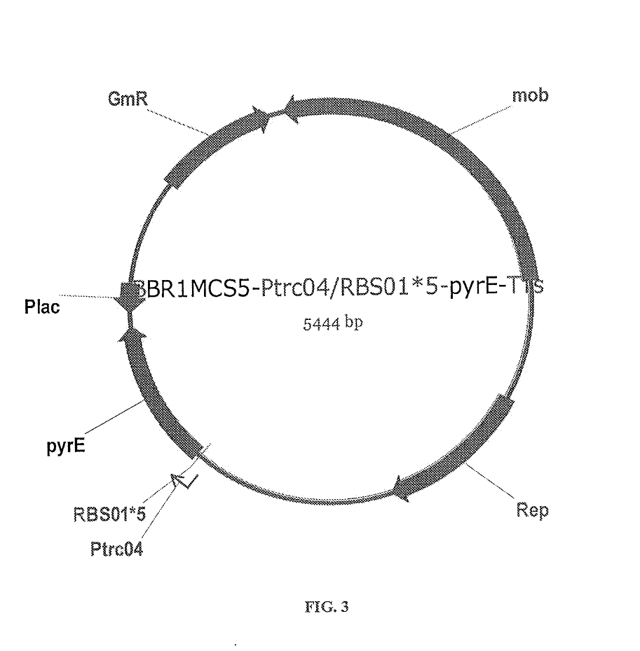

The plasmid pBBR1MCS5-Ptrc04/RBS01*5-pyrE-TTs was constructed from the plasmid pBBR1MCS5 (see M. E. Kovach, (1995), Gene 166:175-176) and pPP1 (see P. Poulsen, (1984), The EMBO Journal 3:1783-1790). The gene pyrE was amplified by PCR from the plasmid pPP1 with the oligonucleotides Ptrc04/RBS01*5-pyrE F and pyrE R including the Ptrc04 promoter and the RBS01*5 in their sequence (Table 1, Seq. N°3 and N°4). The PCR fragment digested with KpnI/EcoRV was cloned into the plasmid pBBR1MCS5 cut by KpnI/SmaI leading to the plasmid pBBR1MCS5-Ptrc04/RBS01*5-pyrE (FIG. 3). The sequence of the recombinant plasmid was checked by DNA sequencing.

### Example 3

### Construction of the Plasmid pBBR1MCS5-Ptrc04/RBS01*5-pyrE-prsA-TTs

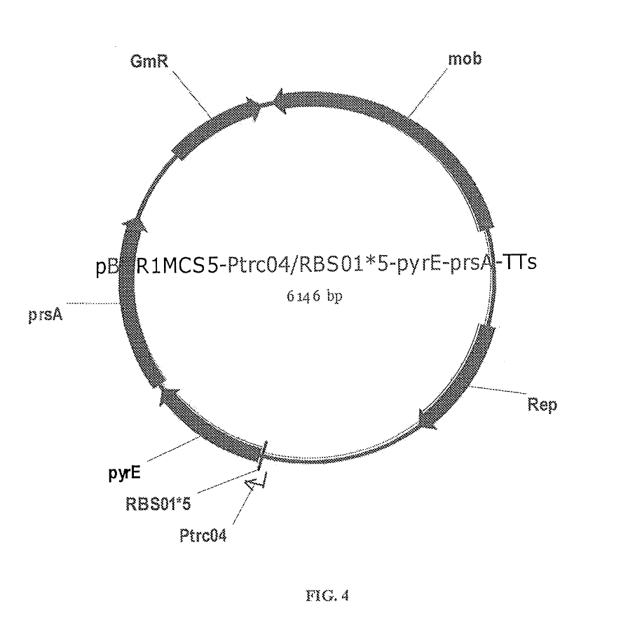

Plasmid pBBR1MCS5-Ptrc04/RBS01*5-pyrE-prsA-TTs was constructed from plasmid pBBR1MCS5-Ptrc04/RBS01*5-pyrE-TTs described above. The gene prsA was amplified by PCR on the MG1655 genomic DNA with the oligonucleotides Oag 0371-prsA F KpnI and Oag 0372-prsA R SmaI given in table 1 (Seq. N°5 and N°6). The PCR fragment digested with SmaI/KpnI and was cloned into the plasmid pBBR1MCS5-Ptrc04/RBS01*5-pyrE-TTs cut by SphI/Klenow/KpnI leading to the plasmid pBBR1MCS5-Ptrc04/RBS01*5-pyrE-prsA-TTs (FIG. 4). The sequence of the recombinant plasmid was checked by DNA sequencing.

### Example 4

### Construction of Strains Producing Glycolic Acid and Overexpressing pyrE with or without prsA: MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01) (pBBR1MCS5-Ptrc04/RBS01*5-pyrE-TTs) and MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01) (pBBR1MCS5-Ptrc04/RBS01*5-pyrE-prsA-TTs)

The strain E. coli MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01) was constructed according to the description given in patent application EP10305635.4.

Plasmids pBBR1MCS5-Ptrc04/RBS01*5-pyrE-TTs and pBBR1MCS5-Ptrc04/RBS01*5-pyrE-prsA-TTs (described in examples 2 and 3 above) were independently introduced into the strain MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01). The resulting strains MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01) (pBBR1MCS5-Ptrc04/RBS01*5-pyrE-TTs) and MG1655 Ptrc50/RBSB/TTG-icd::Cm ΔuxaCA::RN/TTadcca-cI857-PR/RBS01*2-icd-TT02::Km ΔaceB Δgcl ΔglcDEFGB ΔaldA ΔiclR Δedd+eda ΔpoxB ΔackA+pta (pME101-ycdW-TT07-PaceA-aceA-TT01) (pBBR1MCS5-Ptrc04/RB S10*5-pyrE-prsA-TTs) were named AG1629 and AG1630 respectively.

### Example 5

### Construction of Strains Producing Glycolic Acid and Overexpressing pyrE with or without prsA: MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01) (pBBR1MCS5-Ptrc04/RBS01*5-pyrE-TTs) and MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01) (pBBR1MCS5-Ptrc04/RBS01*5-pyrE-prsA-TTs)

The strain E. coli MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01) was constructed according to the description given in patent application EP10305635.4.

The plasmids pBBR1MCS5-Ptrc04/RBS01*5-pyrE-TTs and pBBR1MCS5-Ptrc04/RBS01*5-pyrE-prsA-TTs were independently introduced into the strain MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01). The resulting strains MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01) (pBBR1MCS5-Ptrc04/RBS01*5-pyrE-TTs) and MG1655 TTadcca/cI857/PR01/RBS01*2-icd::Km ΔaceB Δgcl ΔglcDEFGB ΔaldA ΔiclR Δedd+eda ΔpoxB ΔackA+pta ΔaceK::Cm (pME101-ycdW-TT07-PaceA-aceA-TT01) (pBBR1MCS5-Ptrc04/RBS01*5-pyrE-prsA-TTs) were named AG1869 and AG1871 respectively.

### Example 6

### Glycolic Acid Production by Fermentation with Strains that do not Produce Orotate as by-Product

### Strain AG1385:

MG1655 DuxaCA::RN/TTadcca-CI857-PR/RBS01*2-icd-TT02 Ptrc50/RBS05/TTG-icd DaceB Dgcl DglcDEFGB DaldA DiclR Dedd+eda DpoxB DackA+pta (pME101-ycdW*(M)-TT07-PaceA-aceA-TT01).

### Strain AG1629:

MG1655 DuxaCA::RN/TTadcca-CI857-PR/RBS01*2-icd-TT02 Ptrc50/RBS05/TTG-icd DaceB Dgcl DglcDEFGB DaldA DiclR Dedd+eda DpoxB DackA+pta (pME101-ycdW*(M)-TT07-PaceA-aceA-TT01) (pBBR1MCS5-Ptrc04/RBS01*5-pyrE-TTs).

### Strain AG1630:

MG1655 DuxaCA::RN/TTadcca-CI857-PR/RBS01*2-icd-TT02 Ptrc50/RBS05/TTG-icd DaceB Dgcl DglcDEFGB DaldA DiclR Dedd+eda DpoxB DackA+pta (pME101-ycdW*(M)-TT07-PaceA-aceA-TT01) (pBBR1MCS5-Ptrc04/RBS01*5-pyrE-prsA-TTs).

### Strain AG1413:

MG1655 DPicd-CI857-PlambdaR*(−35)/RBS01-icd::Km DaceB Dgcl DglcDEFGB DaldA DiclR Dedd+eda DpoxB DackA+pta DaceK::Cm (pME101-ycdW*(M)-TT07-PaceA-aceA-TT01).

### Strain AG1869:

MG1655 DPicd-CI857-PlambdaR*(−35)/RBS01-icd::Km DaceB Dgcl DglcDEFGB DaldA DiclR Dedd+eda DpoxB DackA+pta DaceK (pME101-ycdW*(M)-TT07-PaceA-aceA-TT01) (pBBR1MCS5-Ptrc04/RBS01*5-pyrE-TTs).

### Strain AG1871:

MG1655 DPicd-CI857-PlambdaR*(−35)/RBS01-icd::Km DaceB Dgcl DglcDEFGB DaldA DiclR Dedd+eda DpoxB DackA+pta DaceK::Cm (pME101-ycdW*(M)-TT07-PaceA-aceA-TT01) (pBBR1MCS5-Ptrc04/RBS01*5-pyrE-prsA-TTs).

### Process of Fermentation

The protocol used for these strains is described in patents applications U.S. 61/245,716 [WDI1] and EP10305635.4.

Precultures were carried out in three 500 ml baffled Erlenmeyer flask filled with 55 ml of synthetic medium MML8AG1_{—}100 (composition in table #3) supplemented with 40 g/l of MOPS and 10 g/l of glucose at 37° C. during 2 days (final optical density of between 7 and 10). 20 mL of this preculture were used for the inoculation of a subculture.

TABLE 3
composition of minimal medium MML8AG1 100.

| Constituent | Concentration (g/l) |
|---|---|
| Citric acid | 6.00 |
| MgSO₄ 7H₂O | 1.00 |
| CaCl₂ 2H₂O | 0.04 |
| CoCl₂ 6H₂O | 0.0080 |
| MnSO₄ H₂O | 0.0200 |
| CuCl₂ 2H₂O | 0.0020 |
| H₃BO₃ | 0.0010 |
| Na₂MoO₄ 2H₂O | 0.0004 |
| ZnSO₄ 7H₂O | 0.0040 |
| Na₂HPO₄ | 2.00 |
| K₂HPO₄ 3H₂O | 10.48 |
| (NH₄)₂HPO₄ | 8.00 |
| (NH₄)₂SO₄ | 5.00 |
| NH₄Cl | 0.13 |
| FeSO₄ 7H₂O | 0.04 |
| Thiamine | 0.01 |

Subcultures were grown in 700 mL working volume vessels mounted on a Multifors Multiple Fermentor System (Infors). Each vessel was filled with 200 ml of synthetic medium MML11AG1_{—}100 (composition in table #3) supplemented with 20 g/l of glucose, 50 mg/l of spectinomycin and was inoculated at an initial optical density of about 1.

TABLE 4
composition of minimal medium MML11AG1 100.

| Constituent | Concentration (g/l) |
|---|---|
| Citric acid | 3.00 |
| MgSO₄ 7H₂O | 1.00 |
| CaCl₂ 2H₂O | 0.04 |
| CoCl₂ 6H₂O | 0.0080 |
| MnSO₄ H₂O | 0.0200 |
| CuCl₂ 2H₂O | 0.0020 |
| H₃BO₃ | 0.0010 |
| Na₂MoO₄ 2H₂O | 0.0004 |
| ZnSO₄ 7H₂O | 0.0040 |
| KH₂PO₄ | 0.70 |
| K₂HPO₄ 3H₂O | 1.17 |
| NH₄H₂PO₄ | 2.99 |
| (NH₄)₂HPO₄ | 3.45 |
| (NH₄)₂SO₄ | 8.75 |
| NH₄Cl | 0.13 |
| FeSO₄ 7H₂O | 0.04 |
| Thiamine | 0.01 |

Cultures were carried out at 30° C. with an aeration of 0.2 lpm and dissolved oxygen was maintained above 30% saturation by controlling agitation (initial: 300 rpm; max: 1200 rpm) and oxygen supply (0 to 40 ml/min) The pH was adjusted to pH 6.8±0.1 by addition of base (mix of NH4OH 7.5% w/w and NaOH 2.5% w/w). The fermentation was carried out in discontinuous fed-batch mode, with a feed stock solution of 700 g/l of glucose (composition in table #5 below).

TABLE 5
composition of feed stock solution.

| Constituent | Concentration (g/l) |
|---|---|
| Glucose | 700.00 |
| MgSO₄ 7H₂O | 2.00 |
| CoCl₂ 6H₂O | 0.0256 |
| MnSO₄ H₂O | 0.0640 |
| CuCl₂ 2H₂O | 0.0064 |
| H₃BO₃ | 0.0032 |
| Na₂MoO₄ 2H₂O | 0.0013 |
| ZnSO₄ 7H₂O | 0.0128 |
| FeSO₄ 7H₂O | 0.08 |
| Thiamine | 0.01 |

When glucose ran out in the culture medium, a pulse of fed restored a concentration of 20 g/l of glucose.

After the 5^{th} pulse of fed (100 g/L of glucose consumed), pH was adjusted to pH 7.4 until the end of the culture. The shift of pH was done in about 2 hours.

Performances of glycolic acid production and accumulation of orotate of strains AG1385, AG1629, AG1630, AG1413, AG1869 and AG1871 grown under these conditions are given in table 6 below.

TABLE 6
Glycolic acid (titre, yield and productivity) and
orotate production of strains AG1385, AG1629,
AG1630, AG1413, AG1869 and AG1871. Mean
values of 2 cultures of each strain are presented.

| | [GA] | Y GA/S | P GA | [orotate] |
|---|---|---|---|---|
| Strain | (g/L) | (g/g) | (g/L/h) | (g/L) |
| AG1385 | 51.3 ± 1.0 | 0.38 ± 0.02 | 0.99 ± 0.07 | 0.8 ± 0.1 |
| AG1629 | 49.9 ± 3.2 | 0.38 ± 0.04 | 0.98 ± 0.04 | 0 |
| AG1630 | 55.1 ± 3.8 | 0.39 ± 0.01 | 1.03 ± 0.04 | 0 |
| AG1413 | 52.5 ± 1.0 | 0.36 ± 0.01 | 1.08 ± 0.07 | 0.7 ± 0.3 |
| AG1869 | 51.1 | 0.38 | 1.13 | 0 |
| AG1871 | 53.6 | 0.39 | 1.19 | 0 |

As can be seen in table 6, overexpression of pyrE gene in strains AG1629, AG1630, AG1869 and AG1871 suppressed orotate accumulation.

It also allowed enhancing glycolic acid production yield in strains AG1869 and AG1871. Performances are better when overproduction of pyrE gene is combined to prsA overexpression.

### Example 7

### Measurement of the Orotate Phospho Ribosyl Transferase (OPRT) Activity

For the determination of Orotate Phospho Ribosyl Transferase (OPRT) activity, cells from flask cultures (25 mg dry weight) were suspended in potassium phosphate buffer and transferred into glass-bead containing tubes for lysis using Precellys (30 s at 6500 rpm, Bertin Technologies). Cell debris was removed by centrifugation at 12000 g (4° C.) during 30 minutes. A Bradford protein assay was used to measure protein concentration. The orotate phosphoribosyl transferase (OPRT) activity present in crude extracts was detected by spectrophotometry at 295 nm (Jasco). The reaction catalyzed by OPRT consists of the transformation of orotate in the presence of AMP into orotidine monophosphate (OMP) and PPi. The assay is based on de measurement of the orotate consumption at 295 nm.

The reaction mixture (1 mL) containing 80 mM of Tris-HCl buffer (pH 8.8), 6 mM MgCl₂, 0.32 mM of orotate and 0.1 to 0.5 μg/μL of crude extract, was incubated at 37° C. during 10 minutes. Then, 0.8 mM of 5-phospho-D-ribosyl-1-diphosphate (PRPP) was added to start the reaction. The activity was calculated using an extinction coefficient of 3.67 M-1.cm⁻¹ at 295 nm for orotate.

Measurement of the Phospho Ribosyl pyrophosphate SynthetAse (PRSA) Activity

For the determination of PRSA activity the cells (25 mg dry weight) from flask cultures were suspended in potassium phosphate buffer and transferred into glass-bead containing tubes for lysis using Precellys (30 s at 6500 rpm, Bertin Technologies). Cell debris was removed by centrifugation at 12000 g (4° C.) during 30 minutes. A Bradford protein assay was used to measure protein concentration. PRSA (PRPP synthetase) activity on ribose-5-phosphate was detected by IC-MS/MS (DIONEX/API2000) by following the production of PRPP. The reaction mixture (1 mL) containing 50 mM of TEA-HCl buffer (pH 7.5), 10 mM MgCl₂, 2 mM of ATP and 2 mM of ribose-5-phosphate, was incubated at 37° C. during 10 minutes. Then, 50 ng of crude extract was added to start the reaction. After 30 minutes, the reaction was stopped by ultrafiltration (Amicon ultra 10K) and the amount of PRPP produced was quantified.

TABLE 7
OPRT and PRSA activities of each strain described in
the previous examples. ND: Not determined.

| | | OPRT | PRSA |
|---|---|---|---|
| Strain | Genotype | (mUI/mg) | (mUI/mg) |
| AG1264 | MG1655 | 11 +/− 6 | ND |
| AG0330 | MG1655 Ptrc50/RBS05/TTG-icd DaceB Dgcl DgIcDEFGB | 8 +/− 5 | ND |
| | DaldA DiclR Dedd+eda (pME101-ycdW-TT07-PaceA-aceA- | | |
| | TT01) | | |
| AG0843 | MG1655 Ptrc50/RBSB/TTG-icd rph+pyrErc DaceB Dgcl | 49 +/− 27 | ND |
| | DglcDEFGB DaldA DiclR Dedd+eda (pME101-ycdW-TT07- | | |
| | PaceA-aceA-TT01) | | |
| AG1385 | MG1655 DuxaCA::RN/TTadcca-Cl857-PR/RBS01*2-icd-TT02 | <8 | ND |
| | Ptrc50/RBS05/TTG-icd DaceB Dgcl DglcDEFGB DaldA DiclR | | |
| | Dedd+eda DpoxB DackA+pta (pME101-ycdW*(M)-TT07- | | |
| | PaceA-aceA-TT01) | | |
| AG1629 | MG1655 DuxaCA::RN/TTadcca-Cl857-PR/RBS01*2-icd-TT02 | 6200 +/− 1436 | ND |
| | Ptrc50/RBS05/TTG-icd DaceB Dgcl DglcDEFGB DaldA DiclR | | |
| | Dedd+eda DpoxB DackA+pta (pME101-ycdW*(M)-TT07- | | |
| | PaceA-aceA-TT01) (pBBR1MCS5-Ptrc04/RBS01*5-pyrE-TTs) | | |
| AG1630 | MG1655 DuxaCA::RN/TTadcca-Cl857-PR/RBS01*2-icd-TT02 | 6529 +/− 2206 | ND |
| | Ptrc50/RBS05/TTG-icd DaceB Dgcl DglcDEFGB DaldA DiclR | | |
| | Dedd+eda DpoxB DackA+pta (pME101-ycdW*(M)-TT07- | | |
| | PaceA-aceA-TT01) (pBBR1MCS5-Ptrc04/RBS01*5-pyrE- | | |
| | prsA-TTs) | | |
| AG1413 | MG1655 DPicd-Cl857-PlambdaR*(−35)/RBS01-icd::Km DaceB | <4 | 17 +/− 2 |
| | Dgcl DglcDEFGB DaldA DiclR Dedd+eda DpoxB DackA+pta | | |
| | DaceK::Cm (pME101-ycdW*(M)-TT07-PaceA-aceA-TT01) | | |
| AG1869 | MG1655 DPicd-Cl857-PlambdaR*(−35)/RBS01-icd::Km DaceB | 7193 +/− 666 | ND |
| | Dgcl DglcDEFGB DaldA DiclR Dedd+eda DpoxB DackA+pta | | |
| | DaceK (pME101-ycdW*(M)-TT07-PaceA-aceA-TT01) | | |
| | (pBBR1MCS5-Ptrc04/RBS01*5-pyrE-TTs) | | |
| AG1871 | MG1655 DPicd-Cl857-PlambdaR*(−35)/RBS01-icd::Km DaceB | 6753 +/− 433 | 103 +/− 11 |
| | Dgcl DglcDEFGB DaldA DiclR Dedd+eda DpoxB DackA+pta | | |
| | DaceK::Cm (pME101-ycdW*(M)-TT07-PaceA-aceA-TT01) | | |
| | (pBBR1MCS5-Ptrc04/RBS01*5-pyrE-prsA-TTs) | | |

### REFERENCES

### Patents

EP 2 025 759 A1
EP 2 025 760 A1
WO2006/069110
WO 2007/141316
U.S. 61/162,712
EP 09155971.6
PCT/EP2006/063046
EP 1529 839A1
EP 1 700 910A2

### Publications

Michihiko Kataoka (2001), Biosci. Biotechnol. Biochem.
Machida, H. and Kuninaka, A. (1969).
Escherichia coli and salmonella typhimurium: cellular and molecular biology”, (1987) Neidhardt, F. C. et al. (eds). American Society for Microbiology, volume 2, chapter 72.
Bachmann, B. J. (1987). Derivations and genotypes of some mutant derivatives of E. coli K-12, p. 1191-1219. In J. L. Ingraham, K. B. Low, B. Magasanik, M. Schaechter, and H. E. Humbarger (ed), Escherichia coli and salmonella typhimurium: cellular and molecular biology.
Jensen K. F. 1993, J. Bacteriol. 175:3401-3707.
Poulsen P. et al. 1984, EMBO 3:1783-1790.
Womack J. E. and O'Donavan G. A. 1978, J. Bacteriol, 136:825-827.
Tsui, H.-C. T. et al. 1991, J. Bacteriol. 173:7395-7400.
Schwartz, M. and Neuhard, J., (1975), J. bacteriol. 121:814-822.
Sambrook et al. (1989) Molecular Cloning: a Laboratory Manual. 2^{nd} ed. Cold Spring Harbor Lab., Cold Spring Harbor, N.Y.
Carrier and Keasling (1998), Biotechnol. Frog. 15, 58-64.

1.-19. (canceled)

20. A method for fermentative production of glycolic acid, and/or a derivative or precursor thereof, comprising:
culturing an Escherichia coli strain in an appropriate culture medium comprising a carbon source, and
recovering produced glycolic acid in the medium,
wherein said Escherichia coli strain is modified to improve conversion of orotate into orotidine 5′-Phosphate.

21. The method of claim 20, wherein the modified strain presents an increased orotate phosphoribosyl transferase specific activity.

22. The method of claim 21, wherein in said modified strain, expression of gene pyrE encoding orotate phosphoribosyl transferase enzyme is increased.

23. The method according to claim 20, wherein the strain is an E. coli K12 strain having a frameshift mutation in the rph-pyrE operon, modified to restore expression of the gene pyrE.

24. The method according to claim 20, wherein the modified strain presents an increased availability of 5-Phosphoribosyl 1-pyrophosphate (PRPP).

25. The method of claim 24, wherein in said modified strain, expression of gene prsA encoding phosphoribosylpyrophosphate synthase is increased.

26. The method according to claim 20, wherein the modified strain is further modified to enhance the production of glycolic acid.

27. The method according to claim 26, wherein the modified strain comprises at least one of the following modifications:
decrease in conversion of glyoxylate to products other than glycolate,
inability to substantially metabolize glycolate,
increase of glyoxylate pathway flux,
increase in conversion of glyoxylate to glycolate,
increase in availability of NADPH.

28. The method according to claim 27, wherein the modified strain comprises at least one of the following modifications:
attenuation of the genes aceB, gleB, gel, eda, attenuation of the genes glcDEFG, aldA,
attenuation of the genes icd, aceK, pta, ackA, poxB, iclR or fadR, and/or overexpression of the gene aceA
overexpression of the genes yedW or yiaE
attenuation of the genes pgi, udhA, edd.

29. The method according to claim 20, wherein the carbon source is at least one selected from group consisting of glucose, sucrose, monosaccharides, oligosaccharides, starch, and glycerol.

30. The method of claim 20, comprising:
a) Fermentation of the modified strain producing glycolic acid
b) Concentration of glycolic acid in bacteria or in the medium and
c) Isolation of glycolic acid from fermentation broth.

31. The method of claim 30 wherein glycolic acid is isolated through polymerization to at least glycolate dimers and recovered by depolymerisation from glycolate dimers, oligomers and/or polymers.

32. An Escherichia coli strain, wherein said strain is modified to improve the conversion of orotate into orotidine 5′-Phosphate.

33. The modified strain of claim 32, wherein the strain presents an increased orotate phosphoribosyl transferase specific activity.

34. The modified strain of claim 33, wherein expression of the gene pyre encoding the orotate phosphoribosyl transferase enzyme is increased.

35. The modified strain of claim 32, wherein the strain is an E. coli K 12 strain having a frameshift mutation in the rph-pyrE operon, and has been modified to restore expression of the gene pyrE.

36. The modified strain of claim 32, wherein the strain presents an increased availability of 5-Phosphoribosyl 1-pyrophosphate (PRPP) as compared with an unmodified strain.

37. The modified strain of claim 36, wherein expression of the gene prsA encoding phosphoribosylpyrophosphate synthase is increased as compared with an unmodified strain.

38. The modified strain of claim 32, wherein the strain is further modified to enhance production of glycolic acid.

39. The modified strain of claim 38, wherein the modified strain comprises at least one of the following modifications:
decrease in conversion of glyoxylate to products other than glycolate,
inability to substantially metabolize glycolate,
increase of glyoxylate pathway flux,
increase in conversion of glyoxylate to glycolate,
increase in availability of NADPH.

40. The modified strain of claim 39 wherein the modified strain comprises at least one of the following modifications:
attenuation of the genes aceB, glcB, gcl, eda,
attenuation of the genes glcDEFG, aldA,
attenuation of the genes icd, aceK, pta, ackA, poxB, iclR or fadR, and/or overexpression of the gene aceA,
overexpression of the genes ycdW or yiaE,
attenuation of the genes pgi, udhA, edd.


SEQUENCE LISTING (8 sequences)

SEQ ID NO: 1 - DNA, 100 bp, Artificial Sequence
  Oligonucleotide
cgccaaactcttcgcgataggccttaaccgccgccagatgttccgccatttccggcttctcttccaggtaagcaatcaggtaatacgactcactataggg

SEQ ID NO: 2 - DNA, 102 bp, Artificial Sequence
  Oligonucleotide
ggtgcgtcccgttaccctgactcgtaactatacaaaacatgcagaaggctcggtgctggtcgaatttggcgataccaaagtgaattaaccctcactaaaggg

SEQ ID NO: 3 - DNA, 85 bp, Artificial Sequence
  Oligonucleotide
gatatcttgaccattaatcatccggctcgtataatgtgtggaataaggaggtatactatgaaaccatatcagcgccagtttattg

SEQ ID NO: 4 - DNA, 27 bp, Artificial Sequence
  Oligonucleotide
ggtaccttaaacgccaaactcttcgcg

SEQ ID NO: 5 - DNA, 29 bp, Artificial Sequence
  Oligonucleotide
ccaggtaccgcatgcctgaggttcttctc

SEQ ID NO: 6 - DNA, 22 bp, Artificial Sequence
  Oligonucleotide
cgggtctttgacccgggttcga

SEQ ID NO: 7 - DNA, 20 bp, Artificial Sequence
  Oligonucleotide
cgacaggttcaaggctacgg

SEQ ID NO: 8 - DNA, 20 bp, Artificial Sequence
  Oligonucleotide
caccaccgatgaaaccctgc